(19) United States
(12) Patent Application Publication
(10) Pub. No.: US 2012/0093935 A1
(43) Pub. Date: Apr. 19, 2012

(54) PARTICLES HAVING A LUMINESCENT INORGANIC SHELL, METHOD FOR COATING PARTICLES AND USE THEREOF

(75) Inventors: Sofia Dembski, Wurzburg, (DE); Carsten Gellermann, Gerbrunn (DE)

(73) Assignee: Fraunhofer-Gesellschaft zur Forderung der angewandten Forschung E.V., Munchen (DE)

(21) Appl. No.: 13/255,722

(22) PCT Filed: Mar. 11, 2010
(86) PCT No.: PCT/EP2010/001543

§ 371 Date: Nov. 8, 2011

(30) Foreign Application Priority Data
Mar. 11, 2009 (DE) 10 2009 012 698.8

(51) Int. Cl. C09K 11/59; A61K 9/14; B05D 5/06; B41M 3/14; B82Y 99/00

(52) U.S. Cl. 424/490; 252/301.4 F; 427/157; 427/7; 427/66; 977/773

(57) ABSTRACT

The invention relates to a method for coating particles with a luminescent inorganic shell. Furthermore, the invention relates to particles having a luminescent inorganic shell and also use thereof.

The invention relates to a method for coating particles with a luminescent inorganic shell. Furthermore, the invention relates to particles having a luminescent inorganic shell and also use thereof.

Inorganic, luminescent nanoparticles are known from prior art. Because of their outstanding optical properties, these have a great potential for application in different fields. Due to their unique physical and chemical properties, these nanomaterials open up diverse possibilities for the production of new functional units for optoelectronics, energy technology or for the field of life science (P. Ravilisety: Mit kleiner Partikelgröβe terbiumaktivierter Yttrium-Gadolinium-Borat-Phosphor und Verfahren zur Herstellung (terbium-activated Yttrium-gadolinium-borate-phosphorus and Method for Production), DE 699 08 107 T2 (2004); R. Lee, Z. Yaniv: Nanoparticle Phosphorus, WO 03/028061 A1 (2003); C. S. Trumble, M. A. Johnson: Luminescent Nanophase Binder Systems for UV and VUV Application, U.S. Pat. No. 0,048,966 A1 (2001); B. Köhler, K. Bohmann, W. Hoheisel, S. Haubold, C. Meyer, T. Heidelberg: Herstellung und Verwendung von in-situ-modifizierten Nanopartikeln (Production and Use of in situ modified Nanoparticles), DE 102 59 935 A1 (2004)).

These particle systems are distinguished above all by an intensive and adjustable luminescence in the visible spectral range. The desired emission colour can be specifically adjusted and controlled by varying the particle size, the composition or by the selection of the crystalline phase (F. Caruso: Colloids and Colloid Assemblies, Wiley-VCH, Weinheim (2004)).

Furthermore, inorganic luminescent nanoparticles have high photostability and therefore offer considerable advantages for long-term investigations in the field of bioanalysis and medical diagnostics (W. Hoheisel, C. Petry, K. Bohmann, M. Haase: Dotierte Nanoteilchen als Biolabel (Doped Nanoparticles as Biolabel), DE 1001 06 643 A1 (2001); W. Chen: Nanoparticle Fluorescence based technology for biological application, J. Nanosci. Nanotechnol., 8 (2008), 1019-1051).

A large number of different possibilities which are already known is available for the production of inorganic luminescent nanoparticles. There may be mentioned here gas-, liquid- and solid phase syntheses, in particular sol-gel technology or organometallic syntheses. The synthesis conditions must thereby be coordinated to each other such that individually present particles with a narrow particle size distribution can be obtained. In the case of many luminescent particle systems, a crystalline material structure is also of particular importance for the optical properties.

One possibility for the production of nanoparticles of a defined form and size and also of a narrow particle size distribution is offered by the sol-gel process (C. Gellermann, H. Wolter: Sphärische oxidische Partikel and deren Verwendung (Spherical Oxidic Particles and Use thereof), DE 100 18 405 B4 (2004)). With the help of this method, luminescent particles based on oxidic materials and layerwise constructed particles with a core-shell structure can be synthesised. The luminescence is thereby achieved by incorporation of organic colourants or rare earth ions (A. Geiger, H. Rupert, K. Kürzinger, P. Sluka, G. Schottner, S. Amberg-Schwab, R. Schwert, H. -P. Josel: Konjugate aus Silicatpartikeln und Biomolekülen und deren Anwendung in der medizinisch-technischen Diagnostik (Conjugates of Silicate Particles and Biomolecules and Application thereof in Medical-Technical Diagnostics), DE 100 47 528 A1 (2002); A. Geiger, D. Griebel, H. Rupert, K. Kürzinger: Modifizierte oxidische Nanopartikel mit hydrophoben Einschlüssen, Verfahren zu ihrer Herstellung und Verwendung dieser Partikel (Modified Oxidic Nanoparticles with Hydrophobic Inclusions, Method for the Production thereof and use of these particles), EP 1 483 203 B1 (2006)).

Because of the low synthesis temperatures, this method is unsuitable for the formation of crystalline particles. Drying and subsequent temperature treatment of wet-chemically-produced particles generally leads to the formation of aggregates.

In the case of gas phase processes, the vapour of the product material is produced by the energy supply by a chemical or physical route and brought for example by cooling into the supersaturated state. This leads to homogeneous nucleation followed by further growth. The formed crystallites can aggregate or agglomerate. Aggregates and agglomerates are produced during the collision and also by the melting (coalescence) of individual particles. By this route, no particles with a homogeneous size distribution can be produced by methods known from prior art. A further disadvantage of this method is the high purity required for the starting materials since purification in the synthesis process can no longer take place. This is associated with high costs (R. Dittmeyer, W. Keim, G. Reysa, A. Oberholz, Chemische Technik: Prozesse und Produkte (Chemical Technology: Processes and Products), Volume 2: Neue Technologie, Wiley-VCH, Weinheim (2004)).

Organometallic synthesis takes an excellent position in the production of crystalline luminescent particles. This method is applied with success for the production of semiconductor nanoparticles and provides monodisperse particles with a diameter of below 10 nm (C. B. Murray, D. J. Norris, M. G. Bawendi: Synthesis and characterization of nearly monodisperse CdE (E=sulphur, selenium, tellurium) semiconductor nanocrystallites, J. Am. Chem. Soc. 115 (1993), 8706-8715; L. H. Qu and X. G. Peng: Control of Photoluminescence Properties of CdSe Nanocrystals in Growth, J. Am. Chem. Soc. 124 (2002), 2049-2055). In this method, high-boiling solvents, such as e.g. phosphines, phosphine oxides, long-chain organic acids and amines, are used, which make possible synthesis temperatures up to approx. 360° C. At the same time, these reagents act as surface stabilisers for control of the particle growth.

The synthesis of inorganic luminescent substances which have no semiconductor properties and the luminescence of which is made possible by the doping of the host material must often be effected via a solid reaction with a multiple hour heat treatment at 500° C. to 1,500° C. or at high pressure in the autoclave (R. Noninger: “Nanoskalige, anorganische Leuchtpigmente und Verfahren zu deren Herstellung” (Nanoscale, Inorganic Luminescent Pigments and Method for the Production thereof), DE 101 11 909 A1, (2002); A. F. Kasenga, A. C. Sigai, T. E. Peters, R. B. Hunt: “Firing and Milling Method for Producing a Manganese Activated Zinc Silicate Phosphorus”, U.S. Pat. No. 4,925,703 (1990); S. Haubold, M. Haase, C. Riwotzki: “Dotierte Nanopartikel” (Doped Nanoparticles), WO 02/20695 A1 (2002); T. S. Amadi, M. Haase, H. Weller: “Low-temperature Synthesis of pure and Mn-doped Willemite Phosphorus (Zn₂SiO₄: Mn) in aqueous Medium”, Mater. Res. Bull. 35 (2000), 1869-1879).

High temperatures and long heating times are required for formation of the host lattice for a sufficiently homogeneous distribution of the luminescent atoms by diffusion in the host lattice.

Furthermore, the inorganic crystal lattice plays an important role here in the case of inorganic luminescent materials. On the one hand, it is a structure-determining network in which doping ions are fixed and, on the other hand, it is also sensitiser for luminescence thereof at the same time. In order to achieve high quantum yields, the doping ions must be situated in as homogeneous and suitable a crystal field as possible. This requires perfect high-quality crystallinity of the matrix lattice. In addition, the donor atoms must be distributed homogeneously. Concentration gradients lead to quenching of the luminescence. High luminescence intensity can only be ensured under these preconditions.

In the last few years, several strategies have been developed for obtaining crystalline luminescent nanoparticles of better quality. For example, particles with a core-shell structure are produced (T. Kazuya, G. Kazuyoshi, F. Naoko, O. Hisatake, H. Hideki: Core/Shell Type Particle Phosphorus, US 2007/0212541 A1 (2007); C. Meyer, M. Haase, W. Hoheisel, K. Bohmann: Kern-Mantel Nanoteilchen für (F)RET-Testverfahren (Core/Shell Nanoparticles for (F)RET Test Methods), DE 603 10 032 T2, (2006); R. Rupaner, R. J. Leyrer, P. Schumacher: Kern/Schale-Partikel, Ihre Herstellung and Verwendung (Core/Shell Particles, their Production and Use), EP 0 955 323 B1 (2004)). The particle core or the shell can thereby have luminescent properties.

The sol-gel process according to M. P. Pechini (M. P. Pechini: Method of Preparing Lead and Alkaline Earth Titanates and Niobates and Coating Methods Using the Same to Form a Capacitor, U.S. Pat. No. 3,330,697, (1967); T. Mayerhöfer, K. F. Renk: Beschichtungsverfahren (Coating Methods), DE 195 46 483 A1 (1997)) serves often as basis for the production of core-shell nanoparticles with an inorganic luminescent shell. In this way, particles with a diameter in the nano- and micrometre range can be provided with a crystalline shell. In the first step, spherical SiO₂ cores are coated wet-chemically with starting compounds for the production of doped luminescent material. In the second step, coated nanoparticles are subjected to a temperature treatment. The heating leads to the formation of a crystalline phase. In this production process, the success quota for obtaining aggregate- or agglomerate-free particles becomes ever smaller with reducing particle size because of the melting of individual particles. Small particles cannot be heated and be aggregate-free without special pretreatment.

In the methods known from prior art, particles which are relatively large and have a wide particle size distribution are often produced, which requires further purification steps, such as e.g. centrifugation. The synthesis of small particles with a narrow size distribution is generally associated with the use of organophosphoric compounds which act as metal complexing agents and, at the same time, as organic reaction medium. Applications of these substances increase the production costs since such substances are expensive. A further disadvantage is the merely limited redispersibility and stability of particles produced in this way in other solvents, especially in aqueous media. Transferring particles into other solvents is associated with exchanging the surface stabilisers, which can often lead to an impairment in the particle properties.

Starting herefrom, it is the object of the present invention to eliminate the disadvantages of the state of the art and to provide a method for coating particles, as a result of which low-aggregate particles with a luminescent inorganic shell can be produced economically and with a narrow size distribution.

This object is achieved by the method having the features of claim 1. Claim 21 concerns particles having a luminescent inorganic shell. Claim 23 is directed towards the use of these particles. Further advantageous embodiments are contained in the dependent claims.

According to the invention, a method for coating particles with an average particle size of 20 nm to 20 μm with a luminescent inorganic shell is provided. This method is effected according to the following steps:
a) synthesis of the cores,
b) coating of the cores with an inorganic doped material,
c) temperature treatment of the coated cores.

The method according to the invention is furthermore characterised in that a step-wise temperature treatment of the coated cores is implemented as step c), with the proviso that the coated cores are pretreated at below 0° C. in at least one first step and then are subjected to a heat treatment, in at least one second step, in order to form a crystalline shell.

The preparation of monodisperse cores can be effected via known wet-chemical methods, e.g. based on the Stöber process or the emulsion- or aerosol method. The modified sol-gel process according to M. P. Pechini can serve as the basis for the coating of amorphous particle cores with a crystalline luminescent shell. The particle cores can hereby be coated by a wet-chemical route with a luminescent material of choice and the doping degree can be varied according to requirement.

The core and the shell can be porous or also dense, according to the particle composition, the particle material and the further application.

There can be used as cores, particles produced in any manner (e.g. based on the Stöber process or the emulsion- or aerosol method), the shape, porosity, size and size distribution of which can be specifically selected according to further applications. Also commercially available SiO₂—and also magnetic particles or cores can be used inter alia.

Inorganic luminescent or electromagnetically active materials are crystalline components which absorb and subsequently emit energy acting on them. The emission of light is termed luminescence. A material which furthermore emits light for longer than 10⁻⁸ s after removal of the excitation source is termed a phosphorescent material. Phosphorescent substances are also known as luminescent materials or luminophores. In contrast to phosphorescent substances, substances, the light emission of which ends immediately or inside 10⁻⁸ s after removal of the excitation source, are termed fluorescent substances. The half-life of the phosphorescence varies as a function of the substance and extends typically from 10⁻⁶ seconds up to days.

Luminescent substances can in principle be termed Stokes (down-converting) or anti-Stokes (up-converting) luminescent substances. Luminescent substances which absorb the energy in the form of a photon of a specific energy and radiate light of a lower energy are called down-converters. In contrast thereto, luminescent substances which absorb energy in the form of two or more photons and consequently emit higher frequencies are termed up-converters. Luminescent substances can furthermore differ or be classified as a function of the origin of the excitation energy. For example, luminescent agents which are excited by low-energy photons are termed photoluminescent and luminescent substances which are excited by means of cathode radiation are termed cathodoluminescent. Other electromagnetically active particles also include pigments and radio frequency-absorbers.

There can be used as coating materials, different inorganic compounds, such as e.g. oxidic compounds, phosphates, sulphides, silicates, aluminates and also mixtures thereof.

Quick-freezing of the coated cores is preferably implemented as pretreatment in the first step. The thus pretreated cores can be freeze-dried subsequently. This leads to a looser arrangement of the particles next to each other and hence prevents melting of the particles during possibly subsequent processes.

In a method variant, the heat treatment for forming the crystalline shell is implemented in steps. Hence the crystalline shell can be formed without or with only a few lattice defects.

There can be used as matrix system for the shell material (host material), for example those subsequently mentioned:

CaS:Ln³⁺ (with Ln³⁺:Ce³⁺, Sm³⁺, Eu²⁺),

silicates Zn₂SiO₄ doped with Mn²⁺ or Ln1, Ln2 (with Ln(1, 2): Ce³⁺, Eu³⁺,

Tb³⁺, Sm³⁺, as alternatives Y₂SiO₅: Eu³⁺, Ce³⁺, Tb³⁺

aluminates (Sr,Ca)Al₂O₄:Ln (with Ln: Ce³⁺, Pr³⁺, Nd³⁺, Eu²⁺/Eu³⁺, Tb³⁺, Dy³⁺)

ZnS: Cu,Pb and various calcium phosphates.

Specific examples of luminescent shell materials are e.g.: LiJ:Eu; NaJ:Tl; CsJ:Tl; CsJ:Na; LiF:Mg; LiF:Mg,Ti; LiF:Mg,Na; KMgF₃:Mn; BaFCl:Eu; BaFCl:Sm; BaFBr:Eu; BaFCl_{0.5}Br_{0.5}:Sm; BaY₂F₈:A (A=Pr, Tm, Er, Ce); BaSi₂O₅:Pb; BaMg₂Al₁₆O₂₇:Eu; BaMgAl₁₄O₂₃:Eu; BaMgAl₁₀O₁₇:Eu; BaMgAl₂O₃:Eu; Ba₂P₂O₇:Ti; (Ba, Zn, Mg)₃Si₂O₇:Pb; Ce(Mg,Ba)Al₁₁O_{19; Ce}_{0.65}Tb_{0.35}MgAl₁₁O₁₉:Ce, Tb; MgAl₁₁O₁₉:Ce, Tb; MgF₂:Mn; MgS:Eu; MgS:Ce; MgS:Sm; MgS(Sm, Ce); (Mg, Ca)S:Eu; MgSiO₃:Mn; 3.5MgO×0.5MgF₂×GeO₂:Mn; MgWO₄:Sm; MgWO₄:Pb; 6MgO×As₂O₅:Mn; (Zn,Mg)F₂:Mn; (Zn₄Be)SO₄:Mn; Zn₂SiO₄:Mn; Zn₂SiO₄:Mn,As; Zn₃(PO₄)₂:Mn; CdBO₄:Mn; CaF₂:Mn; CaF₂:Dy; CaS:A (A=lanthanide, Bi); (Ca,Sr)S:Bi; CaWO₄:Pb; CaWO₄:Sm; CaSO₄:A (A=Mn, lanthanide); 3Ca₃(PO₄)₂×Ca(F,Cl)₂:Sb,Mₙ; CaSiO₃:Mn,Pb; Ca₂Al₂Si₂O₇:Ce; (Ca,Mg)SiO₃:Ce; (Ca,Mg)SiO₃:Ti; 2SrO×6(B₂O₃)×SrF₂:Eu; 3Sr₃(PO₄)₂×CaCl₂:Eu; A₃ (PO₄)₂×ACl₂:Eu (A=Sr, Ca, Ba); (Sr,Mg)₂P₂O₇:Eu; (Sr,Mg)₃(PO₄)₂Sn; SrS:Ce; SrS:Sm,Ce; SrS:Sm; SrS:Eu; SrS:Eu,Sm; SrS:Cu,Ag; Sr₂P₂O₇:Sn; Sr₂P₂O₇:Eu; Sr₄Al₁₄O₂₅:Eu; SrGa₂S₄:A (A=lanthanide, Pb); SrGa₂S₄:Pb; Sr₃Gd₂Si₆O₁₈:Pb,Mn; YF₃:Yb,Er; YF₃:Ln (Ln=lanthanide); YLiF₄:Ln (Ln=lanthanide); Y₃Al₅O₁₂:Ln (Ln=lanthanide); YAI₃(BO₄)₃Nd,Yb; (Y,Ga)BO₃:Eu; (Y,Gd)BO₃:Eu; Y₂Al₃Ga₂O₁₂:Tb; Y₂SiO₅:Ln (Ln=lanthanide); Y₂O₂S:Ln (Ln=lanthanide); YVO₄A (A=lanthanide, In); Y(P,V)O₄:Eu; YTaO₄:Nb; YAIO₃:A (A=Pr, Tm, Er, Ce); YOCl:Yb,Er; LnPO₄:Ce,Tb (Ln=lanthanide or mixture of lanthanides) LuVO₄:Eu; GdVO₄:Eu; Gd₂O₂S:Tb; GdMgB₅O₁₀:Ce,Tb; LaOBr:Tb; La₂O₂S:Tb; LaF₃:Nd,Ce; BaYb₂F₈:Eu; NaYF₄:Yb,Er; NaGdF₄:Yb,Er; NaLaF₄:Yb,Er; LaF₃:Yb,Er,Tm; BaYF₅:Yb,Er; GaN:A (A=Pr, Eu, Er, Tm); Bi₄Ge₃O₁₂; LiNbO₃:Nd,Yb; LiNbO₃:Er; LiCaAlF₆:Ce; LiSrAlF₆:Ce; LiLuF₄:A (A=Pr, Tm, Er, Ce); Li₂B₄O₇:Mn; Y₂O₂Eu; Y₂SiO₅:Eu; CaSiO₃:Ln, wherein Ln=1, 2 or more lanthanides.

With classification according to the host lattice type, the following preferred embodiments are likewise jointly included:
1. Halogenides: e.g. XY₂ (X=Mg, Ca, Sr, Ba; Y=F, Cl, J); CaF₂:Eu(II); BaF₂:Eu; BaMgF₄:Eu; LiBaF₃Eu; SrF₂Eu; SrBaF₂Eu; CaBr₂Eu—SiO₂; CaCJ₂:Eu; CaCJ₂:Eu—SiO₂; CaCJ₂:Eu,Mn—SiO₂; CaJ₂:Eu; CaJ₂:Eu,Mn; KMgF₃:Eu; SrF₂:Eu(II); BaF₂:Eu(II); YF₃; NaYF₄; MgF₂:Mn; MgF₂:Ln (Ln=lanthanide(s))
2. Alkaline earth sulphates: e.g. XSO₄ (X=Mg, Ca, Sr, Ba); SrSO₄:Eu; SrSO₄:Eu,Mn; BaSO₄: Eu; BaSO₄:Eu,Mn; CaSO₄; CaSO₄:Eu; CaSO₄:Eu,Mn; and also mixed alkaline earth sulphates, also in combination with magnesium, e.g. Ca,MgSO₄:Eu,Mn.
3. Phosphates and halophosphates: e.g. CaPO₄:Ce,Mn; Cas(PO₄)₃Cl:Ce,Mn; Cas(PO₄)₃F:Ce,Mn; SrPO₄:Ce,Mn; Srs(PO₄)₃Cl:Ce,Mn; Srs(PO₄)₃F:Ce,Mn; the latter also codoped with Eu(II) or codoped with Eu,Mn; α-Ca₃(PO₄)₂:Eu; β-Ca₃(PO₄)₂:Eu,Mn; Ca₅(PO₄)₃Cl:Eu; Srs(PO₄)₃Cl:Eu; Ba₁₀(PO₄)₆Cl:Eu; Ba₁₀(PO₄)₆Cl:Eu,Mn; Ca₂Ba₃(PO₄)₃Cl:Eu; Ca₅(PO₄)₃F:Eu²⁺X³⁺; Sr₅(PO₄)₃F:Eu²⁺X³⁺(X=Nd, Er, Ho, Tb); Ba₅(PO₄)₃Cl:Eu; β-Ca₃(PO₄)₂:Eu; CaB₂P₂O₉:Eu; CaB₂P₂O₉:Eu; Ca₂P₂O₇:Eu; Ca₂P₂O₇:Eu, Mn; Sr₁₀(PO₄)₆Cl₂:Eu; (Sr,Ca,Ba,Mg)₁₀(PO₄)₆Cl₂:Eu; LaPO₄:Ce; CePO₄; LaPO₄:Eu; LaPO₄:Ce; LaPO₄:Ce,Tb; CePO₄:Tb.
4. Borates: e.g. LaBO₃; LaBO₃:Ce; ScBO₃:Ce; YAIBO₃:Ce; YBO₃:Ce; Ca₂B₅O₉Cl:Eu; xEuO×yNa₂O×zB₂O_{3.}
5. Vanadates: e.g. YVO₄; YVO₄:Eu; YVO_{4:Dy; YVO}₄:Sm; YVO₄:Bi; YVO₄:Bi,Eu; YVO₄:Bi,Dy; YVO₄:Bi,Sm; YVO₄:Tm; YVO₄:Bi,Tm; GdVO₄; GdVO₄:Eu; GdVO₄:Dy; GdVO₄:Sm; GdVO₄:Bi; GdVO₄:Bi,Eu; GdVO₄:Bi,Dy; GdVO₄:Bi,Sm; YVO₄:Eu, YVO₄:Sm; YVO₄:Dy.
6. Aluminates: e.g. MgAl₂O₄:Eu; CaAl₂O₄:Eu; SrAl₂O₄Eu; BaAl₂O₄:Eu; LaMgAl₁₁O₁₉:Eu; BaMgAl₁₀O₁₇:Eu; BaMgAl₁₀O₁₇:Eu,Mn; CaAl₁₂O₁₉:Eu; SrAl₁₂O₁₉:Eu; SrMgAl₁₀O₁₇:Eu; Ba(Al₂O₃)₆:Eu; (Ba,Sr)MgAl₁₀O₁₇:Eu,Mn; CaAl₂O₄:Eu,Nd; SrAl₂O₄:Eu,Dy; Sr₄Al₁₄O₂₅:Eu,Dy.
7. Silicates: e.g. BaSrMgSi₂O₇:Eu; Ba₂MgSiO₇:Eu; BaMg₂Si₂O₇:Eu; CaMgSi₂O₆:Eu; SrBaSiO₄:Eu; Sr₂Si₃O₈×SrCl₂:Eu; Ba₅SiO₄Br₆:Eu; Ba₅SiO₄Cl₆:Eu; Ca₂MgSi₂O₇:Eu; CaAl₂Si₂O₈:Eu; Ca_{1.5}Sr_{0.5}MgSi₂O₇:Eu; (Ca,Sr)₂MgSi₂O₇:Eu; Sr₂LiSiO₄F:Eu; Sr₃Al₂O₆:Eu; Sr₃Al₂O₆:XY (X=Eu, Y=Dy), Sr₅Al₂O₈:Eu, Y₃Al₅O₁₂:Ce, Gd₃Al₅O₁₂: Ce, Lu₃Al₅O₁₂:Ce and (GdLu)₃Al₅O₁₂:Ce, Tb₃Al₅O₁₂:Ce, Tb₃Al₅O₁₂:XY (X=Ce, Y=Eu,Mn).
8. Tungstates and molybdates: e.g. X₃WO₆ (X=Mg, Ca, Sr, Ba); X₂WO₄ (X=Li, Na, K, Rb, Cs); XMoO₄ (X=Mg, Ca, Sr, Ba) and MgWO₄, CaWO₄, CdWO₄, ZnWO₄; as well as polymolybdates or polytungstates or the salts of the corresponding hetero- or isopoly acids.
9. Germanates: e.g. Zn₂GeO₄.
10. In addition, the following classes: ALnO₂:Yb,Er (A=Li, Na; Ln=Gd, Y, Lu); LnAO₄:Yb,Er (Ln=La, Y; A=P, V, As, Nb); Ca₃Al₂Ge₃O₁₂:Er; Gd₂O₂S:Yb,Er; La₂S:Yb,Er, Ba₂ZnS₃:Ce.

A heat treatment at 50° C. to 300° C., preferably at 100° C. to 120° C., is preferably effected in at least one first step. This can then be implemented for gentle removal of volatile components.

In an alternative variant of the method, the heat treatment is effected with mechanical circulation. A more uniform heat distribution is hence made possible.

In at least one second step, a heat treatment for forming the crystalline shell is implemented at a temperature of 400° C. to 1,400° C.

The operation preferably takes place at a heating rate of 50° C. to 500° C., preferably 300° C. to 400° C., per hour. By using higher heating rates, a slow growing together of the particles can thus be avoided. Furthermore, the organic phase can be completely burnt off at high temperatures. Hence this treatment leads to the formation of a crystalline phase in the particle shell and, at the same time, to fixing of the shell to the particle core. Various temperatures can hereby be operated which are required for the formation of the corresponding crystalline phases. The particles are subjected only briefly, for example for 15 minutes, to the actual temperature which is required for the formation of the crystalline phases. Subsequently this is cooled rapidly to room temperature.

The layer thickness can be specifically adjusted by the quantity of starting compounds or by repetition of the already mentioned steps. The particles produced in this way can, according to requirement, easily be redispersed and further used in different solution media, the particle surfaces remaining active.

In a method variant, the temperature required for the formation of the crystalline shell is maintained for 5 minutes to 1.5 hours, preferably for 10 to 30 minutes. These time intervals are varied as a function of the materials used and hence optimum coating results are achieved.

In a preferred method, the layer thickness of the crystalline shell is adjusted to a value of 1 nm to 100 nm. These particles luminesce preferably in the visible spectral range. The luminescence can be detected particularly well in this spectral range.

Furthermore, a further shell which acts as barrier layer can be applied. Foe example, a thin SiO₂—or polymer shell can act as barrier layer. On the one hand, the diffusion of the shell—or doping material from the particle is thus prevented and hence the biocompatibility of the particle systems is increased. On the other hand, variable ligands or spacers can then be coupled more easily thereto. In the case of a silica shell, the coupling can be effected for example by means of silanisation.

Alternatively hereto, a surface functionalisation can be implemented. This is effected preferably by the coupling of ligands to the surface. Hence a covalent surface bonding of polymers and (bio)molecules, e.g. antibodies, can be made possible taking into account the surface affinity.

The surface modification of nanoparticles can basically be effected by two routes. One possibility is the coupling of ligands directly to the particle material. Possibly bifunctional molecules which have an affinity for the particle surface, on the one hand, and the desired functionality, on the other hand, are suitable for this purpose. For the shell material, for example based on silicate, for example bifunctional organosilanes which have the necessary reactive groups can be used for surface derivatisation. Organic ligands are bonded covalently by the reaction between alkoxysilyl units of the silane and hydroxyl groups on the particle surface (silanisation). In the case of other particle systems, suitable surface ligands can be found by having recourse to the high affinity of the surface ions for different functional groups (e.g. Ca²⁺ ions have a high affinity for phosphates and carboxylates or ZnS or CaS can be functionalised with ligands which have a mercapto group).

Another possibility for modifying particles, with respect to setting improved biocompatibility, can be the coating thereof with a thin SiO₂ shell, taking into account the luminescence properties. On the one hand, the diffusion of the doping material from the particle is hence prevented and, on the other hand, biospecific ligands can be coupled more easily to silica. The construction of a thin, stabilising silicon dioxide shell is effected according to known methods. A thin protective layer can be formed by the crosslinking of the organosilanes coupled to the particle surface. The step-wise addition of the silane leads to the controlled construction of the shell, as a result of which a slow shell growth without aggregate formation is made possible. Furthermore, the thickness of the barrier layer can also be adjusted specifically in this way.

In the coating of nanoparticles with a silicon dioxide shell, there often exists the following challenge, that core-shell particles are unstable because of the material properties of the shell or because of the synthesis-caused surface modification in the reaction medium which is suitable for the silicon dioxide growth. In order to stabilise the particles in the required solvent whilst maintaining their properties, these can firstly be coated reversibly with an amphiphilic polymer such as e.g. polyvinylpyrrolidone. Subsequently, following the Stöber process, a silicon dioxide layer can be constructed. The polymer does not thereby take part itself in the reaction and contributes merely to the stabilisation of the particles in the reaction medium. By controlled growth of the silicon dioxide layer, the desired shell thickness can finally be set.

Ligands used for the surface functionalisation are selected from carboxy-, carbonate-, amine-, maleimide-, imine-, imide-, amide-, aldehyde-, thiol-, isocyanate, isothiocyanate-, acylazide-hydroxyl-, N-hydroxysuccinimide ester, phosphate-, phosphonic acid-, sulphonic acid-, sulphochloride, epoxy, CC-double bond-containing units, such as e.g. methacryl- or norbornyl groups. The band width of these ligands makes possible a versatile field of use of these coated particles. On this basis, also different (bio) molecules and polymers can be bonded to the particles. The particles can thus be equipped or coupled with biotin or streptavidin. Hence, a streptavidin-biotin coupling, which has become almost standard in biology, can be implemented with correspondingly functionalised substrates.

The cores can be produced by a wet-chemical route, preferably by the Stöber process or by an emulsion- or aerosol process.

Furthermore, the cores can be produced from oxidic, organic or hybrid materials. Preferably, the cores are produced from silicon dioxide, polystyrene, zirconium oxide, tin oxide, titanium oxide, iron oxide or from hybrid materials. These, possibly amorphous, cores have a particular stability and a uniform spherical shape. Furthermore, a narrow size distribution of the particles is provided here.

Preferably, a wet-chemical process, preferably a sol-gel process, is used for coating the cores with an inorganically-doped material. Advantages of this method are the homogeneous distribution of the educts and, as a consequence thereof, a homogeneous distribution of the doping material. Furthermore, the shell thickness can be specifically adjusted.

In an alternative method variant, metallic salts are mixed with acid and/or polyalcohols and an atomic distribution of the metal cations is produced by the gelling effect. Hence, uniform doping of the material is effected. The starting compound for the shell can be mixed for example with citric acid and polyethyleneglycol, a homogeneous network of metal-chelate complexes being produced firstly. The remaining functional groups of the acid react with the OH groups of the diol to form a polyester. This leads to good statistical distribution of the cations in the mixture and subsequently to the uniform coating of core spheres with the shell material.

Preferably, transition metals, heavy metals or rare earth elements are used as doping materials for the coating. There may be mentioned here by way of example La, Ce, Pr, Sm, Eu, Gd, Tb, Dy, Y, Er, Yb or Mn and also ions thereof. Also Bi and B or the ions thereof can be used as doping materials.

Furthermore, the invention includes particles with a luminescent inorganic shell which can be produced according to the mentioned methods.

For the coating, also oxides or various salts in soluble or colloidal form can be used, such as e.g. acetates, stearates, nitrates, chlorides or phosphates.

Preferably, these particles are agglomerated and/or aggregated to at most <50%, relative to the total weight of the particles. Hence a fine distribution in the solution media is made possible.

According to the invention, the particles having a luminescent inorganic shell are used as luminescent markers for biological and medical diagnostics, as optically detectable diffusion probe, as substrate for heterogeneous catalysis, for the production of light diodes, for the production of safety systems, as marking for detection of counterfeit products and/or originals, as up/down converters, e.g. for solar systems, component for luminescent coating, component for pharmacotherapy (drug-delivery), inks.

With reference to the following examples, the subject according to the invention is intended to be explained in more detail without wishing to restrict said subject to the special embodiments shown here.

### EXAMPLE 1

### Synthesis of SiO₂ Particle Cores

900 ml ethanol and 45 ml aqueous ammonia solution are mixed at 21° C. 45 g tetraethoxysilane (TEOS) are added thereto and agitated. Within 1 h, the solution becomes turbid. The resulting particles are centrifuged and washed twice with ethanol. The average diameter of the cores is 73 nm.

### EXAMPLE 2

### Coating of SiO₂ Cores With a 10 nm Thick Zn₂SiO₄:Mn Shell (5% by mol of Manganese Doping)

31.2 g (142.0 mmol) zinc acetate and 1.3 g (7.5 mmol) manganese acetate are dissolved in a mixture of 837.6 g ethanol and 133.0 g water with the addition of nitric acid (10 mol/l), 62.6 g citric acid (298.0 mmol) and polyethyleneglycol (52 g/l). 6.0 g of the SiO₂ cores described in example 1 are added to the reaction mixture. The batch is agitated at room temperature for 3 h. The coated particles are centrifuged, quick-frozen and subsequently freeze-dried.

The heat treatment includes a pre-drying of the particle powder from 2 to 3 h at 115° C. and 15 minute heating of the sample at 900° C. The heating process is effected at a rate of 300° C./h. Subsequently, the particle sample is cooled rapidly to room temperature. The obtained powder (particle diameter 75 nm) has green luminescence at an excitation wavelength of 254 nm.

### EXAMPLE 3

### Surface Functionalisation With an Aminosilane

1 g of core-shell nanoparticles, described in example 2, are redispersed in 100.00 ml ethanol. 4.3 ml aqueous ammonia solution and 178 μl (692 μmol) N-[3-(trimethoxysilyl)-propyl]diethylenetriamine are added thereto with agitation. Thereafter, the reaction mixture is agitated for 12 h at room temperature. Subsequently, particles are centrifuged off and washed 3 to 4 times with ethanol. The amino functionalisation is detected by means of zeta potential measurement (the isoelectric point is at pH 8.6 to 9.1; for unfunctionalised particles, the isoelectric point is at pH 2.8).

### EXAMPLE 4

### Coating of SiO₂ Cores With a 10 nm Thick Calcium Phosphate Shell (4% by mol of Europium Doping)

0.93 g (3.93 mmol) calcium nitrate tetrahydrate and 29.4 mg (0.08 mmol) europium oxide are dissolved in a mixture of 279.2 g ethanol and 44.3 g water with the addition of nitric acid (10 mol/l), 1.7 g (828.0 mmol) citric acid and polyethyleneglycol (52 g/l). 2.0 g of the SiO₂ cores described in example 1 are added to the reaction mixture. The batch is agitated at room temperature for 3 h. The coated particles are centrifuged, quick-frozen and subsequently freeze-dried.

The heat treatment includes a pre-drying of the particle powder for 1 h at 100° C. and 15 min heating of the sample at 800° C. The heating process is effected at a rate or heating rate of 300° C. per hour. Subsequently, the particle sample is cooled rapidly to room temperature. The obtained powder (particle diameter 80 nm) luminesces with a pink colour at an excitation wavelength of 254 nm.

1. A method for coating particles with an average particle size of 20 nm to 20 μm with a luminescent inorganic shell, said method comprising:
(a) synthesizing cores,
(b) coating the cores with an inorganic doped material, and
(c) treating the coated cores with a step-wise temperature treatment of the coated cores comprising at least one first step of pretreating the coated cores at below 0° C. and at least one second step heat treating the coated cores in order to form a crystalline shell.

2. The method according to claim 1, wherein pretreating the coated cores comprises a quick-freezing of the coated cores.

3. The method according to claim 2, wherein the at least one first step further comprises freeze drying the pretreated cores.

4. The method according to claim 1, wherein heat treating the coated cores to form the crystalline shell is implemented in steps.

5. The method according to claim 1, wherein the heat treating is effected at 50° C. to 300° C., preferably at 100° to 120° C.

6. The method according to claim 1, wherein the heat treating is effected with mechanical circulation.

7. The method according to claim 4, wherein the heat treating for forming the crystalline shell is implemented at a temperature of 400° C. to 1,400° C.

8. The method according to claim 7, wherein the heat treating takes place at a heating rate of 50° C. to 500° C., preferably 300° C. to 400° C., per hour.

9. The method according to claim 7, wherein the heat treating comprises maintaining the temperature for the formation of the crystalline shell for 5 min to 1.5 hours, preferably for 10 to 30 minutes.

10. The method according to claim 1, wherein the layer thickness of the crystalline shell is adjusted to a value of 1 nm to 100 nm.

11. The method according to claim 1, further comprising applying a further shell which acts as barrier layer.

12. The method according to claim 1, wherein a surface functionalisation is implemented.

13. The method according to claim 12, wherein the surface functionalisation is effected by the coupling of ligands.

14. The method according to claim 12, wherein the ligands used for the surface functionalisation are selected from carboxy-, carbonate-, amine-, maleimide-, imine-, imide-, amide-, aldehyde-, thiol-, isocyanate, isothiocyanate-, acylazide-hydroxyl-, N-hydroxysuccinimide ester, phosphate-, phosphonic acid-, sulphonic acid-, sulphochloride, epoxy, CC-double bond-containing units, such as e.g. methacryl- or norbornyl groups.

15. The method according to claim 1, wherein the cores are produced by a wet-chemical route, preferably by the Stöber process or by an emulsion- or aerosol process.

16. The method according to claim 15, wherein the cores are produced from oxidic, organic or hybrid materials.

17. The method according to claim 1, wherein the cores are produced from silicon oxide, polystyrene, zirconium oxide, tin oxide, titanium oxide, iron oxide or from hybrid materials.

18. The method according to claim 1, wherein coating the cores with the inorganic doped material comprises a wet-chemical process, preferably a sol-gel process, for coating the cores.

19. The method according to claim 18, wherein the wet-chemical process comprises mixing metallic salts with acid and/or polyalcohols and an atomic distribution of the metal cations is produced by a gelling effect.

20. The method according to claim 1, wherein transition metals, heavy metals or rare earth elements are used as doping materials for inorganic doped material.

21. Particles having a luminescent inorganic shell produced according to the method of claim 1.

22. The particles having a luminescent inorganic shell according to claim 21, wherein the particles are agglomerated and/or aggregated to at most <50%, relative to the total weight of the particles.

23. The method according to claim 1, further comprising, after step (c), using the coated cores as luminescent markers for biological and medical diagnostics, as optically detectable diffusion probe, as substrate for heterogeneous catalysis, for the production of light diodes, for the production of safety systems, as marking for the detection of counterfeit products and/or originals, as up/down converters, as a component for luminescent coating, as a component for pharmacotherapy, or as an ink.
